SEQUENCE LISTING (17 sequences)

SEQ ID NO: 1 - DNA, 3066 bp, Proteus vulgaris
  CDS (1)..(3066)
atgccgatatttcgttttactgcacttgcaatgacattggggctattatcagcgccttataacgcgatggcagccaccagcaatcctgcatttgatcctaaaaatctgatgcagtcagaaatttaccattttgcacaaaataacccattagcagacttctcatcagataaaaactcaatactaacgttatctgataaacgtagcattatgggaaaccaatctcttttatggaaatggaaaggtggtagtagctttactttacataaaaaactgattgtccccaccgataaagaagcatctaaagcatggggacgctcatctacccccgttttctcattttggctttacaatgaaaaaccgattgatggttatcttactatcgatttcggagaaaaactcatttcaaccagtgaggctcaggcaggctttaaagtaaaattagatttcactggctggcgtgctgtgggagtctctttaaataacgatcttgaaaatcgagagatgaccttaaatgcaaccaatacctcctctgatggtactcaagacagcattgggcgttctttaggtgctaaagtcgatagtattcgttttaaagcgccttctaatgtgagtcagggtgaaatctatatcgaccgtattatgttttctgtcgatgatgctcgctaccaatggtctgattatcaagtaaaaactcgcttatcagaacctgaaattcaatttcacaacgtaaagccacaactacctgtaacacctgaaaatttagcggccattgatcttattcgccaacgtctaattaatgaatttgtcggaggtgaaaaagagacaaacctcgcattagaagagaatatcagcaaattaaaaagtgatttcgatgctcttaatattcacactttagcaaatggtggaacgcaaggcagacatctgatcactgataaacaaatcattatttatcaaccagagaatcttaactcccaagataaacaactatttgataattatgttattttaggtaattacacgacattaatgtttaatattagccgtgcttatgtgctggaaaaagatcccacacaaaaggcgcaactaaagcagatgtacttattaatgacaaagcatttattagatcaaggctttgttaaagggagtgctttagtgacaacccatcactggggatacagttctcgttggtggtatatttccacgttattaatgtctgatgcactaaaagaagcgaacctacaaactcaagtttatgattcattactgtggtattcacgtgagtttaaaagtagttttgatatgaaagtaagtgctgatagctctgatctagattatttcaataccttatctcgccaacatttagccttattattactagagcctgatgatcaaaagcgtatcaacttagttaatactttcagccattatatcactggcgcattaacgcaagtgccaccgggtggtaaagatggtttacgccctgatggtacagcatggcgacatgaaggcaactatccgggctactctttcccagcctttaaaaatgcctctcagcttatttatttattacgcgatacaccattttcagtgggtgaaagtggttggaataacctgaaaaaagcgatggtttcagcgtggatctacagtaatccagaagttggattaccgcttgcaggaagacacccttttaactcaccttcgttaaaatcagtcgctcaaggctattactggcttgccatgtctgcaaaatcatcgcctgataaaacacttgcatctatttatcttgcgattagtgataaaacacaaaatgaatcaactgctatttttggagaaactattacaccagcgtctttacctcaaggtttctatgcctttaatggcggtgcttttggtattcatcgttggcaagataaaatggtgacactgaaagcttataacaccaatgtttggtcatctgaaatttataacaaagataaccgttatggccgttaccaaagtcatggtgtcgctcaaatagtgagtaatggctcgcagctttcacagggctatcagcaagaaggttgggattggaatagaatgcaaggggcaaccactattcaccttcctcttaaagacttagacagtcctaaacctcataccttaatgcaacgtggagagcgtggatttagcggaacatcatcccttgaaggtcaatatggcatgatggcattcgatcttatttatcccgccaatcttgagcgttttgatcctaatttcactgcgaaaaagagtgtattagccgctgataatcacttaatttttattggtagcaatataaatagtagtgataaaaataaaaatgttgaaacgaccttattccaacatgccattactccaacattaaataccctttggattaatggacaaaagatagaaaacatgccttatcaaacaacacttcaacaaggtgattggttaattgatagcaatggcaatggttacttaattactcaagcagaaaaagtaaatgtaagtcgccaacatcaggtttcagcggaaaataaaaatcgccaaccgacagaaggaaactttagctcggcatggatcgatcacagcactcgccccaaagatgccagttatgagtatatggtctttttagatgcgacacctgaaaaaatgggagagatggcacaaaaattccgtgaaaataatgggttatatcaggttcttcgtaaggataaagacgttcatattattctcgataaactcagcaatgtaacgggatatgccttttatcagccagcatcaattgaagacaaatggatcaaaaaggttaataaacctgcaattgtgatgactcatcgacaaaaagacactcttattgtcagtgcagttacacctgatttaaatatgactcgccaaaaagcagcaactcctgtcaccatcaatgtcacgattaatggcaaatggcaatctgctgataaaaatagtgaagtgaaatatcaggtttctggtgataacactgaactgacgtttacgagttactttggtattccacaagaaatcaaactctcgccactcccttga

SEQ ID NO: 2 - protein, 1021 aa, Proteus vulgaris
MPIFRFTALAMTLGLLSAPYNAMAATSNPAFDPKNLMQSEIYHFAQNNPLADFSSDKNSILTLSDKRSIMGNQSLLWKWKGGSSFTLHKKLIVPTDKEASKAWGRSSTPVFSFWLYNEKPIDGYLTIDFGEKLISTSEAQAGFKVKLDFTGWRAVGVSLNNDLENREMTLNATNTSSDGTQDSIGRSLGAKVDSIRFKAPSNVSQGEIYIDRIMFSVDDARYQWSDYQVKTRLSEPEIQFHNVKPQLPVTPENLAAIDLIRQRLINEFVGGEKETNLALEENISKLKSDFDALNIHTLANGGTQGRHLITDKQIIIYQPENLNSQDKQLFDNYVILGNYTTLMFNISRAYVLEKDPTQKAQLKQMYLLMTKHLLDQGFVKGSALVTTHHWGYSSRWWYISTLLMSDALKEANLQTQVYDSLLWYSREFKSSFDMKVSADSSDLDYFNTLSRQHLALLLLEPDDQKRINLVNTFSHYITGALTQVPPGGKDGLRPDGTAWRHEGNYPGYSFPAFKNASQLIYLLRDTPFSVGESGWNNLKKAMVSAWIYSNPEVGLPLAGRHPFNSPSLKSVAQGYYWLAMSAKSSPDKTLASIYLAISDKTQNESTAIFGETITPASLPQGFYAFNGGAFGIHRWQDKMVTLKAYNTNVWSSEIYNKDNRYGRYQSHGVAQIVSNGSQLSQGYQQEGWDWNRMQGATTIHLPLKDLDSPKPHTLMQRGERGFSGTSSLEGQYGMMAFDLIYPANLERFDPNFTAKKSVLAADNHLIFIGSNINSSDKNKNVETTLFQHAITPTLNTLWINGQKIENMPYQTTLQQGDWLIDSNGNGYLITQAEKVNVSRQHQVSAENKNRQPTEGNFSSAWIDHSTRPKDASYEYMVFLDATPEKMGEMAQKFRENNGLYQVLRKDKDVHIILDKLSNVTGYAFYQPASIEDKWIKKVNKPAIVMTHRQKDTLIVSAVTPDLNMTRQKAATPVTINVTINGKWQSADKNSEVKYQVSGDNTELTFTSYFGIPQEIKLSPLP

SEQ ID NO: 3 - protein, 18 aa, Artificial Sequence
  Description of Artificial Sequence Synthetic
ATSNPAFDPKNLMQSEIY

SEQ ID NO: 4 - DNA, 21 bp, Artificial Sequence
  Description of Artificial Sequence Synthetic
ctaatgggttattttgtgcaa

SEQ ID NO: 5 - RNA, 17 nt, Artificial Sequence
  modified base 3, 6, 9 and 15: N is A, T, C or G
gcnacnucnaayccngc

SEQ ID NO: 6 - DNA, 17 bp, Artificial Sequence
  modified base 3, 6 and 15: N is A, T, C or G
gcnacnagyaayccngc

SEQ ID NO: 7 - RNA, 17 nt, Artificial Sequence
  modified base 9: N is A, T, C or G
uacguyagncuyuadau

SEQ ID NO: 8 - RNA, 17 nt, Artificial Sequence
  Description of Artificial Sequence Synthetic
uacguyucrcuyuadau

SEQ ID NO: 9 - DNA, 25 bp, Artificial Sequence
  Description of Artificial Sequence Synthetic
catttgatcctaaaaatctgatgca

SEQ ID NO: 10 - DNA, 18 bp, Artificial Sequence
  Description of Artificial Sequence Synthetic
gcaaccagcaatcctgca

SEQ ID NO: 11 - DNA, 20 bp, Artificial Sequence
  Description of Artificial Sequence Synthetic
gactacgtcaggcttttaat

SEQ ID NO: 12 - DNA, 53 bp, Artificial Sequence
  Description of Artificial Sequence Synthetic
gcaaccagcaatcctgcatttgatcctaaaaatctgatgcagtccgaaattta

SEQ ID NO: 13 - DNA, 53 bp, Artificial Sequence
  Description of Artificial Sequence Synthetic
taattttcggactgcatcagatttttaggatcaaatgcaggattgctggttgc

SEQ ID NO: 14 - DNA, 122 bp, Proteus vulgaris
gatatcactcaatcattaaatttaggcacaacgatgggctatcagcgttatgacaaatttaatgaaggacgcattggtttcactgttagccagcgtttctaaggagaaaaataatgccgata

SEQ ID NO: 15 - DNA, 60 bp, Phaseolus vulgaris
atgacaaatttaatgaaggacgcattggtttcactgttagccagcgtttctaaggagaaa

SEQ ID NO: 16 - DNA, 400 bp, Proteus vulgaris
  CDS (296)..(400)
cagactgcttatggcaaattaaccccctctcttaatcttcgttattcaaagatattgcaggtgacaatgatatcaatcaacgccacagccttacctattttaatacagggggaagtacctttgatattaaaggaaataccgttggtggtgacattattagtgcggaattaggtgcaaatctcgatatcactcaatcattaaatttaggcacaacgatgggctatcagcgttatgacaaatttaatgaaggacgcattggtttcactgttagccagcgtttctaaggagaaaaataatgccgatatttcgttttactgcacttgcaatgacattggggctattatcagcgccttataacgcgatggcagccaccagcaatcctgcatttgatgctaaaaat

SEQ ID NO: 17 - protein, 35 aa, Proteus vulgaris
MPIFRFTALAMTLGLLSAPYNAMAATSNPAFDAKN

(19) United States
(12) Patent Application Publication
(10) Pub. No.: US 2011/0027860 A1
(43) Pub. Date: Feb. 3, 2011

(54) GENE ENCODING CHONDROITINASE ABC AND USES THEREFOR

(75) Inventors: Nobuyuki SATO, Ibaraki (JP); Masahiko SHIMADA, Ibaraki (JP); Hiroshi ODA, Ibaraki (JP)

Correspondence Address: NELSON MULLINS RILEY & SCARBOROUGH LLP;FLOOR 30, SUITE 3000, ONE POST OFFICE SQUARE, BOSTON, MA, 02109, US

(73) Assignee: MARUHA NICHIRO SEAFOODS, INC., Tokyo (JP)

(21) Appl. No.: 12/712,960

(22) Filed: Feb. 25, 2010

Related U.S. Application Data
(63) Continuation of application No. 11/313,169, filed on Dec. 20, 2005, now abandoned.
Application No. 11/313,169 is a continuation of application No. 08/488,960, filed on Jun. 7, 1995, now Pat. No. 7,008,783.
Application No. 08/488,960 is a continuation of application No. 08/184,435, filed on Jan. 14, 1994, now abandoned.
Application No. 08/184,435 is a division of application No. 08/074,349, filed on Jun. 8, 1993, now abandoned.

(30) Foreign Application Priority Data
Feb. 24, 1993 (JP) 5-35810

(51) Int. Cl. C12N 9/88; C07H 21/00; C12N 15/63; C12N 5/10

(52) U.S. Cl. 435/232; 536/23.2; 435/320.1; 435/325

(57) ABSTRACT

Nucleic acid sequences coding for the chondroitinase ABC gene and isolated chondroitinase ABC protein produced in a host cell transformed with a nucleic acid vector directing the expression of a nucleotide sequence coding for chondroitinase ABC protein are described. Chondroitinase ABC prepared by chemical synthesis is also described. Monoclonal and polyclonal antibodies which are specifically reactive with chondroitinase ABC protein are disclosed. The isolated chondroitinase ABC can be used in methods of treating intervertebral disc displacement, promoting neurite regeneration, and detecting galactosaminoglycans.

### RELATED APPLICATIONS

This application is a continuation application of U.S. patent application Ser. No. 11/313,169, filed on Dec. 20, 2005, which is a continuation application of U.S. patent application Ser. No. 08/488,960, filed on Jun. 7, 1995, which is a continuation application of U.S. patent application Ser. No. 08/184,435, filed on Jan. 14, 1994, which is a divisional application of U.S. patent application Ser. No. 08/074,349, filed on Jun. 8, 1993, which claims priority to Japanese Patent Application No. 5-35810, filed on Feb. 24, 1993. The entire contents of each of the aforementioned applications and all references, issued patents, and published patent applications cited therein are incorporated herein by reference.

### BACKGROUND OF THE INVENTION

Chondroitin lyase (EC 4.2.2.4) or chondroitinase ABC is an enzyme which catalyzes the depolymerization of chondroitin sulfate. Through β-elimination of 1,4 hexosaminidic bonds, chondroitinase ABC degrades chondroitin, chondroitin 4-sulfate (chondroitin A sulfate), dermatan sulfate (chondroitin B sulfate), chondroitin 6-sulfate (chondroitin C sulfate) and hyaluronate to the respective unsaturated disaccharides (Δdi-OS for chondroitin, Δdi-4S for chondroitin A sulfate, Δdi-4-6S for chondroitin B sulfate and Δdi-6S for chondroitin C sulfate, respectively). The enzyme has been isolated in various strains of bacteria (Neuberg, C. et al., (1914) Biochem. Z. 67: 82-89) (Neuberg, C. et al. (1931) Biochem. Z. 234: 345-346; Yamagata, T. et al., (1968) J. Biol. Chem. 243: 1523-1535) including Proteus vulgaris (Yamagata, T. et al. (1968) J. Biol. Chem. 243: 1523-1535; Thurston, C. F. (1974) J. Gen. Microbiol. 80: 515-522; Sato N. et al. (1986) Agric. Biol. Chem. 50: 1057-1059; Sato N. et al. (1986) Biotechnol. Bioeng. 28: 1707-1712; Sato, N. et al. (1986) J. Ferment. Technol. 64: 155-159).

Chondroitin sulfate consists of alternating β1-3 glucuronidic and β1-4 N-acetylgalactosaminidic bonds, and is sulfated at either C-4 or C-6 of the N-acetylgalactosamine pyranose. Chondroitin sulfate is known to be widely distributed in mammalian tissue, such as in skin, cornea, bone and especially in cartilage. Thus, chondroitinase ABC has been used as an experimental reagent for the determination or quantitation of total amount of galactosaminoglycans in the field of orthopedic surgery (Linker, A. et al. (1960) J. Biol. Chem. 235: 3061-3065; Saito, H. et al. (1968) J. Biol. Chem. 243: 1536-1542; Pettipher, E. R. et al. (1989) Arthritis Rheum. 32: 601-607; Caterson, B. et al. (1990) J. Cell Science 97: 411-417; and Seibel, M. J. et al. (1992) Arch. Biochem. Biophys. 296: 410-418).

Recently, chondroitinase ABC has been reported to be a potential reagent for chemonucleolysis, an established treatment for intervertebral disc displacement (Kato, F. et al. (1990) Clin. Orthop. 253: 301-308; Henderson, N. et al. (1991) Spine 16: 203-209). However, for the utilization of chondroitinase ABC as a clinical reagent, there are many problems to be overcome. For example, the preparation of chondroitinase ABC from P. vulgaris requires tedious and intricate procedures, since the cellular content of the enzyme is low. Therefore, an efficient method for the efficient preparation of highly purified chondroitinase ABC is now sought.

### SUMMARY OF THE INVENTION

This invention pertains to nucleic acid sequences coding for the chondroitinase ABC gene and isolated chondroitinase ABC protein produced in a host cell transformed with a nucleic acid vector directing the expression of a nucleotide sequence coding for chondroitinase ABC. Chondroitinase ABC prepared by chemical synthesis is also provided. This invention further provides monoclonal and polyclonal antibodies which are specifically reactive with chondroitinase ABC. The isolated chondroitinase ABC can be used in methods of treating intervertebral disc displacement and promoting neurite regeneration or in method of detecting the presence of galactosaminoglycans.

### BRIEF DESCRIPTION OF THE DRAWINGS

FIGS. 1-A and 1-B show the primers used for polymerase chain reaction (PCR) amplification of chondroitinase ABC from P. vulgaris genomic DNA; FIG. 1-B also shows the probe used for plaque hybridization; and FIG. 1-C shows the restriction maps for three recombinant phages and the fragment of phage 11-5 which was subcloned into pSTV29 for sequencing.

FIG. 2 shows the construction of pCHSP, a hybrid plasmid containing the putative promoter region of chondroitinase ABC (SEQ ID NO:14).

FIG. 3 shows primer extension analysis using a sequencing ladder (SEQ ID NO:15).

FIG. 4 shows the nucleotide sequence of the promoter region of chondroitinase ABC (SEQ ID NO: 16) and the peptide sequence (SEQ ID NO:17).

FIG. 5 shows the construction of plasmids pCHS 6, pCHSΔ 6, and pCHS 26 each of which contains a fragment of the chondroitinase ABC gene.

FIG. 6 shows SDS-PAGE and immunoblot analysis of recombinant chondroitinase ABC protein produced by pCHSΔ 6 transformed E. coli (lane 1); protein produced by pSTV 29 without the chondroitinase ABC gene in E. coli (lane 2); natural chondroitinase ABC produced by P. vulgaris (lane 3); and molecular weight markers (lane 4).

FIG. 7 shows the DNA (SEQ ID NO:1) and amino acid sequence (SEQ ID NO:2) of the chondroitinase ABC gene.

### DETAILED DESCRIPTION OF THE INVENTION

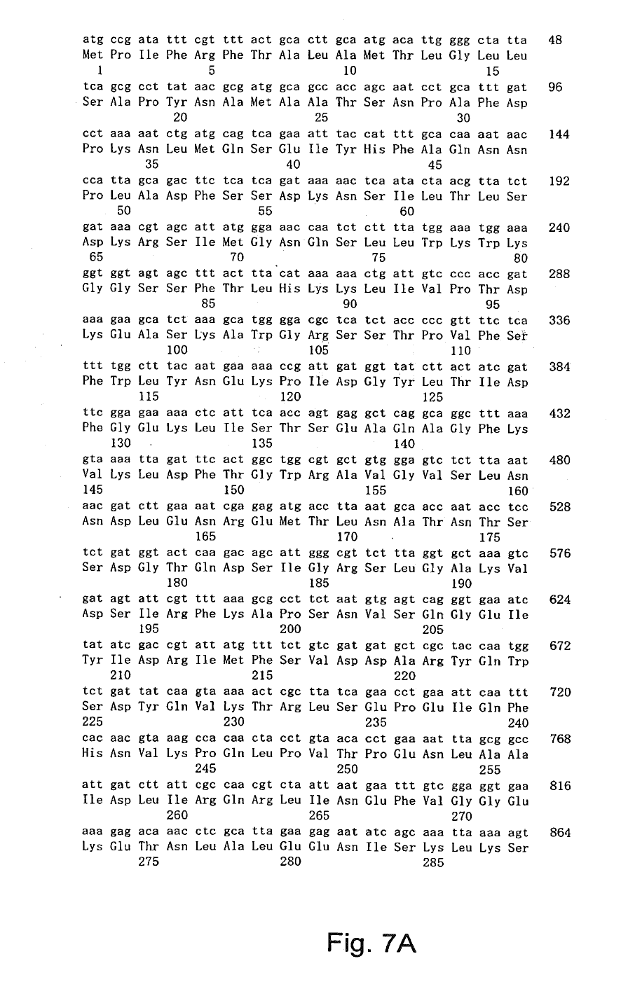

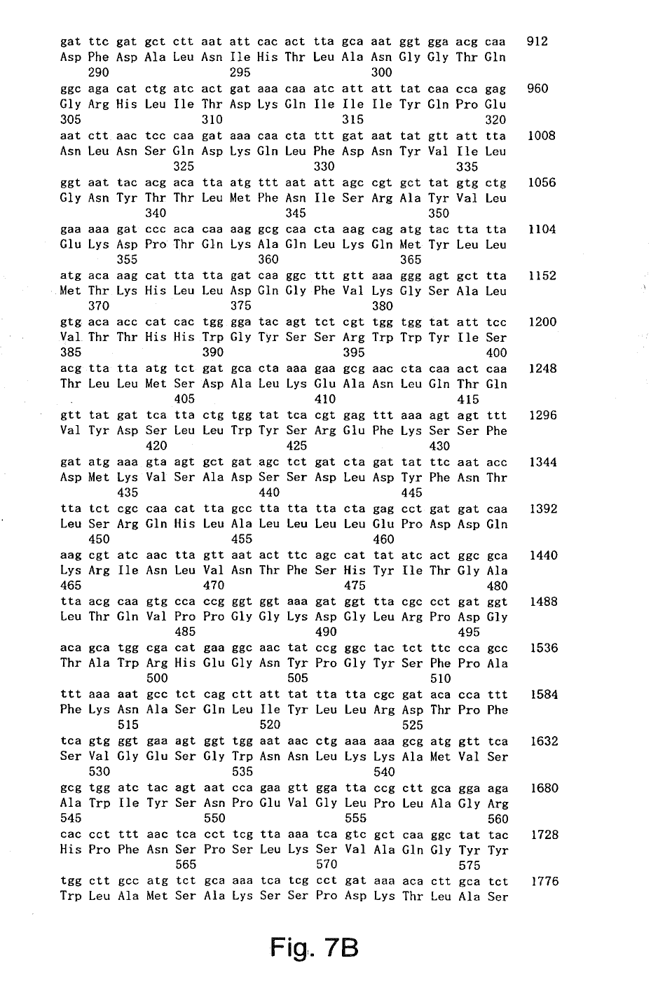

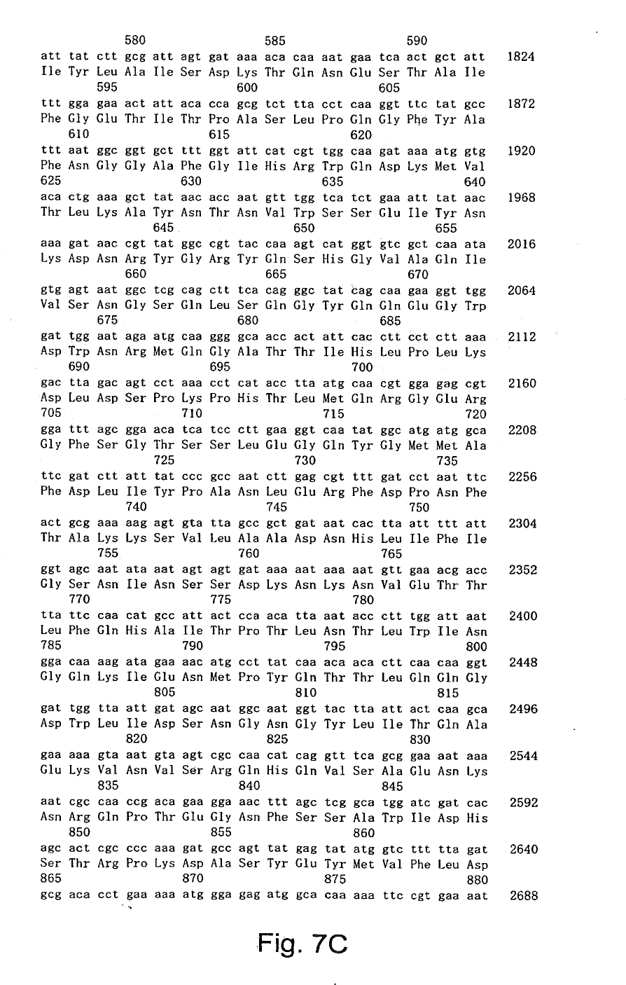

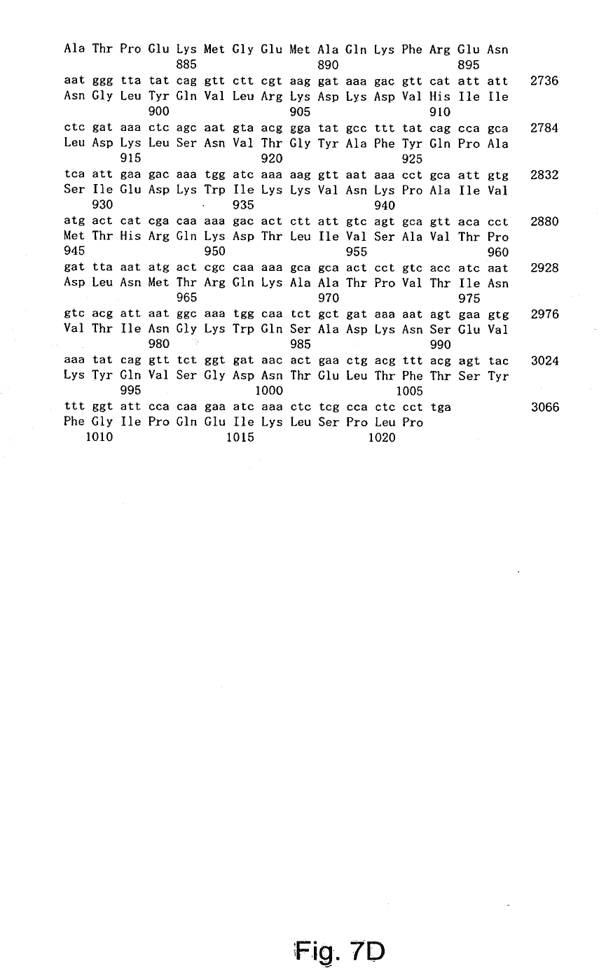

This invention pertains to nucleic acid sequences coding for chondroitinase ABC, an enzyme which degrades chondroitin A, B, and C. The chondroitinase ABC gene was derived using recombinant DNA techniques. A nucleic acid sequence coding for chondroitinase ABC preferably has the sequence shown in SEQ ID NO:1 (FIG. 7). The deduced amino acid sequence of chondroitinase ABC is shown in SEQ ID NO:2 (FIG. 7).

Accordingly, one aspect of the invention pertains to an isolated nucleic acid having a nucleotide sequence coding for chondroitinase ABC, fragments thereof, or equivalents thereof. The term nucleic acid as used herein is intended to include such fragments or equivalents. A nucleic acid sequence coding for chondroitinase ABC can obtained from mRNA present in Proteus vulgaris. Nucleic acid sequences coding for chondroitinase ABC can also be obtained from P. vulgaris genomic DNA. The nucleic acid sequence coding for chondroitinase ABC can be obtained using the method disclosed herein or any other suitable technique for isolation and molecular cloning of genes. The nucleic acid sequences of the invention can be DNA or RNA. The preferred nucleic acid is a DNA having the sequence depicted in SEQ ID NO:1 (FIG. 7) or equivalents thereof.

The term equivalent is intended to include nucleotide sequences coding for functionally equivalent chondroitinase ABC proteins. For example, DNA sequence polymorphisms within the nucleotide sequence of chondroitinase ABC (especially those within the third base of a codon) may result in “silent” mutations which do not affect the amino acid sequence of the chondroitinase ABC protein. However, it is expected that DNA sequence polymorphisms that do lead to changes in the amino acid sequence of chondroitinase ABC will exist. It will be appreciated by one skilled in the art that these variations in one or more nucleotides (up to about 3-4% of the nucleotides) of the nucleic acid sequence coding for chondroitinase ABC may exist due to natural allelic variation. Any and all such nucleotide variations and resulting amino acid polymorphisms are within the scope of the invention. Furthermore, there may be one or more isoforms or related, cross-reacting family members of chondroitinase ABC. Such isoforms or family members are defined as proteins related in function and amino acid sequence to chondroitinase ABC, but encoded by genes at different loci.

A fragment of the nucleic acid sequence coding for chondroitinase ABC is defined as a nucleotide sequence having fewer nucleotides than the nucleotide sequence coding for the entire amino acid sequence of chondroitinase ABC protein. Such fragments encode a catalytically-active fragment of chondroitinase ABC protein which depolymerizes chondroitin A, B, or C. Nucleic acid fragments within the scope of the invention include those capable of hybridizing with nucleic acid from other animal species for use in screening protocols to detect chondroitinase ABC or enzymes that are cross-reactive with chondroitinase ABC. Nucleic acid sequences within the scope of the invention may also contain linker sequences, modified restriction endonuclease sites and other sequences useful for molecular cloning, expression or purification of recombinant chondroitinase ABC or catalytically-active fragments thereof.

This invention also provides expression vectors containing a nucleic acid sequence coding for chondroitinase ABC, operably linked to at least one regulatory sequence. Operably linked is intended to mean that the nucleotide sequence is linked to a regulatory sequence in a manner which allows expression of the nucleotide sequence. Regulatory sequences are art-recognized and are selected to direct expression of chondroitinase ABC. Accordingly, the term regulatory sequence includes promoters, enhancers and other expression control elements. Such regulatory sequences are described in Goeddel, Gene Expression Technology: Methods in Enzymology 185, Academic Press, San Diego, Calif. (1990). It should be understood that the design of the expression vector may depend on such factors as the choice of the host cell to be transformed and/or the type of protein desired to be expressed.

This invention further pertains to a host cell transformed to express chondroitinase ABC. The host cell may be any prokaryotic or eukaryotic cell. For example, chondroitinase ABC protein may be expressed in bacterial cells such as E. coli, insect cells (baculovirus), yeast, or mammalian cells such as Chinese hamster ovary cells (CHO). Other suitable host cells may be found in Goeddel, (1990) supra or one known to those skilled in the art.

Expression in eukaryotic cells such as mammalian, yeast, or insect cells can lead to partial or complete glycosylation and/or formation of relevant inter- or intra-chain disulfide bonds of recombinant protein. Examples of vectors for expression in yeast S. cerivisae include pYepSec1 (Baldari. et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of proteins in cultured insect cells (SF 9 cells) include the pAc series (Smith et al., (1983) Mol. Cell. Biol. 3:2156-2165) and the pVL series (Lucklow, V. A., and Summers, M. D., (1989) Virology 170:31-39). Generally COS cells (Gluzman, Y., (1981) Cell 23:175-182) are used in conjunction with such vectors as pCDM 8 (Aruffo, A. and Seed, B., (1987) Proc. Natl. Acad. Sci. USA 84:8573-8577) for transient amplification/expression in mammalian cells, while CHO (dhfr⁻ Chinese Hamster Ovary) cells are used with vectors such as pMT2PC (Kaufman et al. (1987), EMBO J. 6:187-195) for stable amplification/expression in mammalian cells. Vector DNA can be introduced into mammalian cells via conventional techniques such as calcium phosphate or calcium chloride co-precipitation, DEAE-dextran-mediated transfection, or electroporation. Suitable methods for transforming host cells can be found in Sambrook et al., Molecular Cloning: A Laboratory Manual, 2nd Edition, Cold Spring Harbor Laboratory press (1989), and other laboratory textbooks.

Expression in prokaryotes is most often carried out in E. coli with either fusion or non-fusion inducible expression vectors. Fusion vectors usually add a number of NH₂ terminal amino acids to the expressed target gene. These NH₂ terminal amino acids often are referred to as a reporter group. Such reporter groups usually serve two purposes: 1) to increase the solubility of the target recombinant protein; and 2) to aid in the purification of the target recombinant protein by acting as a ligand in affinity purification. Often, in fusion expression vectors, a proteolytic cleavage site is introduced at the junction of the reporter group and the target recombinant protein to enable separation of the target recombinant protein from the reporter group subsequent to purification of the fusion protein. Such enzymes include Factor Xa, thrombin and enterokinase. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-tranferase, maltose E binding protein, or protein A, respectively, to the target recombinant protein.

Inducible non-fusion expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET 11d relies on transcription from the T7 gn10-lac 0 fusion promoter mediated by coexpressed viral RNA polymerase (T7 gn1). This viral polymerase is supplied by host strains BL21(DE3) or HMS174(DE3) from a resident g prophage harboring a T7 gn1 under the transcriptional control of the lacUV 5 promoter.

One strategy to maximize recombinant chondroitinase ABC expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy would be to alter the nucleic acid sequence of the chondroitinase ABC gene to be inserted into an expression vector so that the individual codons for each amino acid would be those preferentially utilized in highly expressed E. coli proteins (Wada et al., (1992) Nuc. Acids Res. 20:2111-2118). Such alteration of nucleic acid sequences of the invention can be carried out by standard DNA synthesis techniques.

The nucleic acid sequences of the invention can also be chemically synthesized using standard techniques. Various methods of chemically synthesizing polydeoxynucleotides are known, including solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

This invention further pertains to methods of producing chondroitinase ABC protein. For example, a host cell transformed with a nucleic acid vector directing expression of a nucleotide sequence coding for chondroitinase ABC protein can be cultured under appropriate conditions to allow expression of chondroitinase ABC to occur. The protein may be secreted and isolated from a mixture of cells and medium containing chondroitinase ABC protein. Alternatively, the protein may be retained cytoplasmically and the cells harvested, lysed and the protein isolated. The culture includes host cells, media and other byproducts. Suitable mediums for cell culture are well known in the art. Chondroitinase ABC protein can be isolated from cell culture medium, host cells, or both using techniques known in the art for purifying proteins including ion-exchange chromatography, gel filtration chromatography, ultrafiltration, electrophoresis, and immunoaffinity purification with antibodies specific for chondroitinase ABC or fragment thereof.

Another aspect of the invention pertains to isolated chondroitinase ABC protein. The term “chondroitinase ABC” or “chondroitinase ABC protein” is intended to include functional equivalents thereof and catalytically-active fragments thereof. The term functional equivalent is intended to include proteins which differ in amino acid sequence from the chondroitinase ABC sequence depicted in SEQ ID NO:2 (FIG. 7) but where such differences result in a modified protein which functions in the same or similar manner as chondroitinase ABC or which has the same or similar characteristics of chondroitinase ABC. For example, a functional equivalent of chondroitinase ABC may have a modification such as a substitution, addition or deletion of an amino acid residue which is not directly involved in the enzyme activity of chondroitinase ABC (i.e., the ability of chondroitinase ABC to depolymerize chondroitin 4-sulphate, chondroitin 6-sulfate, and dermatan sulfate). Various modifications of the chondroitinase ABC protein to produce functional equivalents of chondroitinase ABC are described in detail herein.

The term isolated as used herein refers to chondroitinase ABC protein substantially free of cellular material or culture medium when produced by recombinant DNA techniques, or chemical precursors or other chemicals when chemically synthesized. Such chondroitinase ABC protein is also characterized as being essentially free of all other P. vulgaris proteins. Accordingly, an isolated chondroitinase ABC protein is produced recombinantly or synthetically and is substantially free of cellular material and culture medium or substantially free of chemical precursors or other chemicals and is essentially free of all other P. vulgaris proteins.

Fragments of chondroitinase ABC which depolymerize chondroitin A, B, or C (referred to herein as catalytically-active fragments) may be obtained, for example, by screening peptides recombinantly produced from the corresponding fragment of the nucleic acid sequence of chondroitinase ABC coding for such peptides. In addition, fragments can be chemically synthesized using techniques known in the art such as by conventional Merrifield solid phase f-Moc or t-Boc chemistry. For example, the chondroitinase ABC protein may be arbitrarily divided into fragments of desired length. The fragments can be produced (recombinantly or by chemical synthesis) and tested to determine their enzymatic activity, for example, by contacting the fragment with chondroitin A, B, or C under conditions which allow for depolymerization and determining the extent to which depolymerization occurs.

It is possible to modify the structure of the chondroitinase ABC protein for such purposes as increasing solubility, enhancing therapeutic efficacy, or stability (e.g., shelf life ex vivo and resistance to proteolytic degradation in vivo). Such modified proteins or analogues are considered functional equivalents of the chondroitinase ABC protein as defined herein.

To facilitate purification and potentially increase solubility of the chondroitinase ABC protein, it is possible to add an amino acid reporter group to the protein backbone. For example, hexa-histidine can be added to the protein for purification by immobilized metal ion affinity chromatography (Hochuli, E. et al., (1988) Bio/Technology 6:1321-1325). In addition, to facilitate isolation of chondroitinase ABC protein free of irrelevant sequences, specific endoprotease cleavage sites can be introduced between the sequences of the reporter group and the protein or peptide.

Another aspect of the invention pertains to an antibody specifically reactive with chondroitinase ABC. The antibodies of this invention can be used to isolate the naturally-occurring or native form of chondroitinase ABC or to neutralize the enzyme so that it is unable to depolymerize chondroitin. For example, by using isolated chondroitinase ABC protein based on the cDNA sequence of chondroitinase ABC, anti-protein/anti-peptide antisera or monoclonal antibodies can be made using standard methods. A mammal such as a mouse, a hamster or a rabbit can be immunized with an immunogenic form of the isolated chondroitinase ABC protein (e.g., chondroitinase ABC protein or an antigenic fragment which is capable of eliciting an antibody response). Techniques for conferring immunogenicity on a protein or peptide include conjugation to carriers or other techniques well known in the art. The chondroitinase ABC protein or fragment thereof can be administered in the presence of adjuvant. The progress of immunization can be monitored by detection of antibody titers in plasma or serum standard ELISA or other immunoassay can be used with the immunogen as antigen to assess the levels of antibodies.

Following immunization, anti-chondroitinase ABC antisera can be obtained and, if desired, polyclonal anti-chondroitinase ABC antibodies isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, for example the hybridoma technique originally developed by Kohler and Milstein, Nature (1975) 256:495-497, as well as other techniques such as the human B-cell hybridoma technique (Kozbar et al., Immunology Today (1983) 4:72) and the EBV-hybridoma technique to produce human monoclonal antibodies (Cole et al., Monoclonal Antibodies and Cancer Therapy (1985) Alan R. Liss, Inc. pp. 77-96). Hybridoma cells can be screened immunochemically for production of antibodies specifically reactive with the chondroitinase ABC protein and the monoclonal antibodies isolated.

The term antibody as used herein is intended to include fragments thereof which are also specifically reactive with the chondroitinase ABC protein or fragment thereof. Antibodies can be fragmented using conventional techniques and the fragments screened for utility in the same manner as described above for whole antibodies. For example, F(ab′)₂ fragments can be generated by treating the antibody with pepsin. The resulting F(ab′)₂ fragment can be treated with papain to reduce disulfide bridges to produce Fab′ fragments. The antibody of the present invention is further intended to include bispecific and chimeric molecules having an anti-chondroitinase ABC portion.

This invention provides therapeutic compositions for the treatment of intervertebral displacement or nerve damage. The composition comprises a therapeutically active amount of chondroitinase ABC protein and a pharmaceutically acceptable carrier. Administration of the therapeutic compositions of the present invention to an individual to be treated can be carried out using known procedures, at dosages and for periods of time effective to depolymerize chondroitin A, B, or C. A therapeutically active amount of chondroitinase ABC protein may vary according to factors such as the amount of chondroitin to be eliminated, the age, sex, and weight of the individual, and the ability of the chondroitinase ABC protein to depolymerize the chondroitin. Dosage regima may be adjusted to provide the optimum therapeutic response.

The active compound (i.e., chondroitinase ABC protein) may be administered in a convenient manner such as by injection (subcutaneous, intravenous, etc.). If the active compound is administered by injection, for example, about 100 units of active compound (i.e., chondroitinase ABC protein) per dosage unit may be administered to treat intervertebral disc displacement. One unit is the amount of enzyme needed to mediate the release of one micromole of 4,5 unsaturated disaccharide from a substrate of chondroitin C sulfate per minute at 37° C., pH 6.0.

The active compound may be administered parenterally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate or gelatin.

Sterile injectable solutions can be prepared by incorporating active compound (i.e., chondroitinase ABC protein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., protein) plus any additional desired ingredient from a previously sterile-filtered solution thereof.

As used herein “pharmaceutically acceptable carrier” includes any and all solvents, dispersion media, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic composition is contemplated. Supplementary active compounds can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the elimination of chondroitin A, B, or C.

Isolated chondroitinase ABC protein (i.e., chondroitinase ABC produced recombinantly or by chemical synthesis) is essentially free of all other P. vulgaris proteins. Such protein is of a consistent, well-defined composition and biological activity for use in preparations which can be administered for therapeutic purposes (e.g., to treat intervertebral disc displacement). Such proteins can also be used as diagnostic reagents or in the study of the mechanism of chondroitinase ABC and to design modified derivatives or analogs useful in the depolymerization of chondroitin.

This invention also provides a method of treating intervertebral disc displacement by chemonucleolysis using isolated chondroitinase ABC. Chondroitinase ABC is a particularly useful enzyme for the selective chemonucleolysis of the nucleus pulposus (See, for example, U.S. Pat. No. 4,696,816). The nucleus pulposus is made up of proteoglycans and collagen fibers. Chondroitinase ABC attacks the polysaccharide side chains of the proteoglycans and reduces the swelling of the disc without affecting the structural collagen components or degrading the protein element of the proteoglycan. The disc then shrinks and pressure on the spinal cord is relieved. Thus, to treat intervertebral disc displacement, an active amount of the chondroitinase ABC protein of the invention can be applied to the affected area. For example, 100 units of isolated chondroitinase ABC can be injected into the center of a disc by the standard technique of intradiscal injection (Brown, Intradiscal Therapy, Year Book Medical Publishers, Inc., Chicago, 1983).

The invention further provides a method of treating nerve damage by applying an active amount of the chondroitinase ABC protein of the invention to the affected area to degrade chondroitin 6-sulfate proteoglycans. It has been found that chondroitin 6-sulfate proteoglycans inhibit regeneration of neurites in the adult vertebrate central nervous system (McKeon et al., J. Neurosci 11:3398-3411 (1991)). By removing chondroitin 6-sulfate proteoglycans from the point of injury, it is possible to promote neurite regeneration. For example, a therapeutically effective amount of isolated chondroitinase ABC can be applied to the point of injury in an individual to degrade inhibitory chondroitin 6-sulfate proteoglycans. More than one dose may be administered as indicated by the exigencies of the therapeutic situation.

The chondroitinase ABC protein of the invention can also be used as a diagnostic reagent for detecting the presence of a galactosaminoglycan, such as chondroitin sulfate. For example, the chondroitinase ABC protein can be used as a reagent for determining or quantitating the amount of galactosaminoglycan in a mammalian tissue, such as skin, cornea, bone or cartilage (See e.g., Linker, A. et al. (1960) J. Biol. Chem. 235: 3061-3065; Saito, H. et al. (1968) J. Biol. Chem. 243: 1536-1542; Pettipher, E. R. et al. (1989) Arthritis Rheum. 32: 601-607; Caterson, B. et al. (1990) J. Cell Science 97: 411-417; and Seibel, M. J. et al. (1992) Arch. Biochem. Biophys. 296: 410-418). To determine the presence of chondroitin sulfate in a mammalian tissue, chondroitinase ABC protein can be contacted with a sample of the tissue and the presence or amount of chondroitin sulfate determined using methods well known in the art.

The invention is further illustrated by the following examples which should not be construed as further limiting the subject invention. The contents of all references and published patent applications cited throughout this application are hereby incorporated by reference. The following methods and materials were used throughout the examples discussed below.

### Materials and Methods

Bacterial strains, plasmid and phage P. vulgaris IFO3988 was provided by the Institute for Fermentation, Osaka, Japan. E. coli P2392 (hsdR514 (r^{k-}, m^{k+}), supE44, supF58, lacY1 or (lacIZY), galT22, metB1, trpR55, (P2)) was used as the lysogen for P2 phage. EMBL3 vector was purchased from Toyobo Co., Ltd., Japan. PCR products were ligated with pT7 Blue T-vector (Takara Shuzo Co., Ltd., Japan). E. coli JM109 (recA1, endA1, gyrA96, thi, hsdR17 (r^{k-}, m^{k+}), supE44, relA1, λ-, Δ(lac-proB), (F′, proAB, lacIq M15, traD36) was used as the host strain for pMC1871 promoter selection vector (Pharmacia LKB, Japan). E. coli XL1-Blue (endA1, hsdR17 (r^{k-}, m^{k+}), supE44, thi-1, recA1, gyrA96, relA1, Δ(lac), (F′, proAB, lacI, (lacZΔM15, Tn10 (tet^{r})) (Int'l Dep. No. FERM BP-4170). E. coli XL1-Blue is a host cell for both pSTV28, and pSTV29 (Takara Shuzo Co., Ltd., Japan).

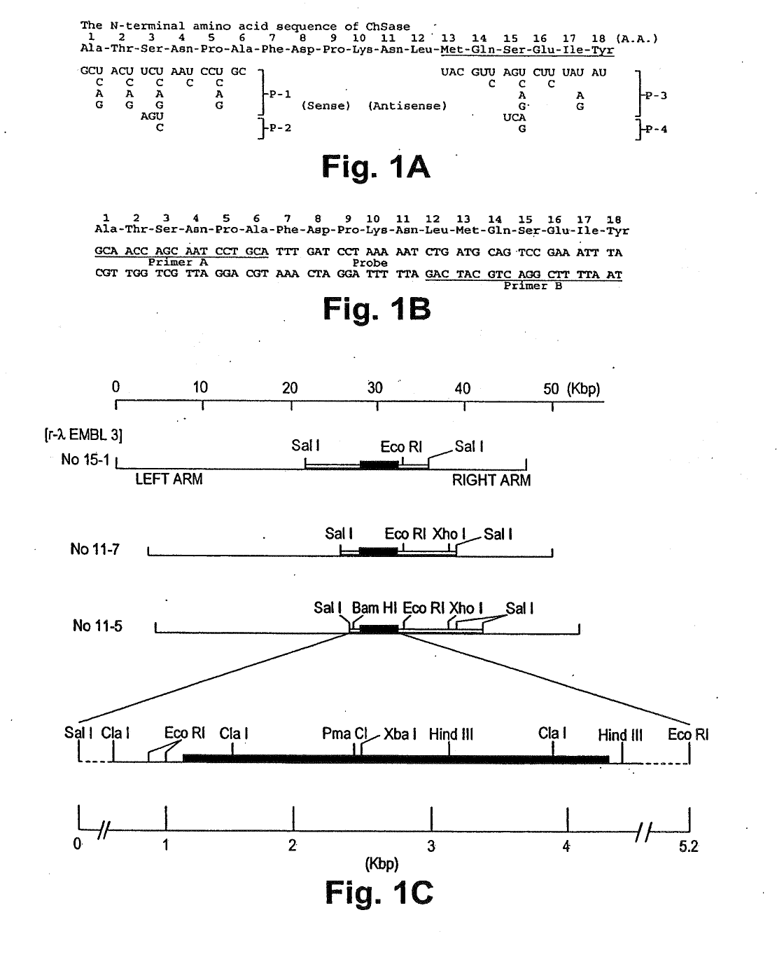

N-terminal amino acid sequence Chondroitinase ABC was purified as described previously (Sato, N. et al. (1986) Agric. Biol. Chem. 50: 1057-1059). The N-terminal amino acid sequence of chondroitinase ABC was sequenced by automatic Edman degradation on a gas-phase sequencer (Applied Biosystem, Foster, Calif.). The sequence of the N-terminal region of chondroitinase ABC was Ala-Thr-Ser-Asn-Pro-Ala-Phe-Asp-Pro-Lys-Asn-Leu-Met-Gln-Ser-Glu-Ile-Tyr (18 amino acid residues) (SEQ ID NO:3) The double stranded DNA sequence is shown in FIG. 1-B (SEQ ID NOS:12-13).

Isolation of DNA and synthesis of nucleic acid, primer, and probe Isolation of chromosomal DNA of P. vulgaris was carried out by the standard method (Silhavy, T. J. et al. (1984) Experiments with Gene Fusion, Cold Spring Harbor Laboratory Press). Oligonucleotides used as primers and probe were synthesized with the DNA synthesizer, Cyclone Plus (Milligene/Biosearch, Bedford, Mass.).

Construction and screening of the gene library SauIII AI-partially digested fragments of total DNA were ligated to the BamHI site in λEMBL3 arms according to Frischauf et al. (J. Mol. Biol. 170: 827-842 (1983)). The ligation mixture was packaged in vitro and transfected to E. coli P2392 according to the instructions of the suppliers (Stratagene, La Jolla, Calif.).

PCR amplification Primers for the chondroitinase ABC gene were designed according to the amino acid sequence of the chondroitinase ABC N-terminal region (SEQ ID NO:3)(FIG. 1-A). The primers were as follows 5′-GCNACNUCNAAYCCNGC-3′ (P-1, sense) (SEQ ID NO:5); 5′-GCNACNAGYAAYCCNGC-3′ (P-2, sense) (SEQ ID NO:6); 5′-UACGUYAGNCUYUADAU-3′ (P-3, antisense) (SEQ ID NO:7); 5′-UACGUYUCRCUYUADAU-3′ (P-4 antisense) (SEQ ID NO:8) (FIG. 1-A). PCR was performed using a GeneAmp Kit (Takara Shuzo Co., Ltd., Japan) in a final volume of 100 μl which contained: 1 μg of genomic DNA solution, 10 μl of 10×PCR reaction buffer, 16 μl of 1.25 mM dNTP mixture, 0.6 nmol of mixed primers and 2.5 units of Taq DNA polymerase (Takara Shuzo Co., Ltd., Japan). The mixture was subjected to PCR amplification using the DNA thermal cycler (GeneAmp PCR System 9600, Perkin-Elmer/Cetus, Norwalk, Conn.) for 28 cycles. Each cycle was 1 minute at 93° C. (denaturation), 1.5 minutes at 50° C. (annealing) and 0.5 minute at 72° C. (elongation). PCR products were analyzed by electrophoresis through a 5% agarose gel (Nusieve GTG agarose, FMC Bioproducts, Rockford, Me.) and the 54 bp fragment encoding 17 amino acids of N-terminal region was cut out of the gel. Gel-purified PCR products were directly cloned into pT7 Blue PCR vector.

DNA sequencing and isolation of the chondroitinase ABC gene Double-stranded plasmids purified by polyethylene glycol were denatured with alkali and sequenced by dideoxynucleotide chain termination method (Sanger, R. et al. (1977) Proc. Natl. Acad. Sci. USA 74: 5463-5467) using the sequence system, Hitachi WS10A Personal Sequencer (Hitachi Electronics Co., Ltd., Japan) Direct sequencing was done according to the method of Gyllesten & Erlich (Gyllensten, U. (1989) in PCR Technology, Erlich, H. A., Ed., Stockton Press, New York, pp. 45-60). PCR screening was carried out by the method of Olson et al. (Science 245: 1434-1435 (1989)). Plaque hybridization (Sambrook, J. et al. (1989) Molecular Cloning: A Laboratory Manual, 2nd ed., Cold Spring Harbor Laboratory Press) and Southern hybridization (Southern, E. M. (1975) J. Mol. Biol. 8: 503-517) were performed as outlined in the instructions of the supplier (Amersham Japan).

Primer extension analysis A 21-mer oligonucleotide (5′-CTA ATG GGT TAT TTT GTG CAA-3′) (SEQ ID NO:4) complementary to the 5′-end (nucleotides 355-375) of the chondroitinase ABC gene was used as a primer. It was labeled with γ-³²P ATP (Amersham Japan) using polynucleotide kinase (Toyobo Co., Ltd., Japan). Total RNA of P. vulgaris was prepared according to the method of Aiba (J. Biol. Chem. 260: 3063-3070 (1985)). The labeled primer and 5 μg of total RNA were coprecipitated with ethanol. After annealing at 25° C. for 6 hours in a hybridization buffer (80% formamide, 40 mM PIPES (pH 6.4), 1 mM EDTA and 400 mM NaCl), 250 mM NaCl, 50 mM sodium acetate (pH 4.6), 4.5 mM ZnSO₄, 100 μg/ml heat-denatured salmon testes DNA and 15 unit/μl reverse transcriptase of Rous associated virus 2 (Takara Shuzo Co., Ltd., Japan) were added to the mixture. The primer extension reaction was carried out at 37° C. for 60 minutes.

Culture conditions Cells of E. coli XL1-Blue carrying recombinant plasmid were grown in 3 ml of LB broth (1% tryptone, 0.5% yeast extract, 1% NaCl, 25 μg/ml of chloramphenicol (pH 7.5)) at 37° C. for 16 hr with reciprocation (120 rpm, 5 cm stroke). The cells were harvested by centrifugation and washed twice with 0.85% saline solution. Cells were transferred to 100 ml of chondroitin 6-sulfate (Taiyo Fishery Co., Ltd., Japan) medium (0.7% K₂HPO₄, 0.3% KH₂PO₄, 0.01% MgSO₄.7H₂O, 0.1% (NH₄)₂SO₄, 0.1% yeast extract, 0.3% chondroitin 6-sulfate, 0.01% glucose, 25 μg/ml chloramphenicol (pH 7.5)) or glucose medium (composition is the same as that of chondroitin medium except that glucose (0.3%) was used as a carbon source) to make a final concentration of A₆₁₀=0.05. After incubation for 3 days at 37° C. with reciprocation, the cells were removed by centrifugation and degradation products of chondroitin 6-sulfate in the culture fluid were determined. The cells harvested from chondroitin and glucose media were washed twice with 50 mM Tris-HCl buffer (pH 8.0) and sonicated at 90 kHz for 5 minutes at 0° C. The cell debris were removed by centrifugation at 20,000 g for 30 minutes, and the supernatant was used for the assay of chondroitinase ABC.

Enzyme assay Chondroitinase ABC was assayed as described previously (Sato, N. et al. (1986) J. Ferment. Technol. 64: 155-159). The assay mixture (3 ml) containing 0.5% chondroitin 6-sulfate, 100 mM potassium phosphate buffer (pH 8.0) and cell extract, was incubated at 37° C. for 10 minutes, and the amount of N-acetylgalactosamine end group formed was determined by the method of Reissig (J. Biol. Chem. 217: 959-966 (1955)). Activity was expressed as the quantity of enzyme that catalyzed the formation of 1 μmol of unsaturated disaccharide (Δdi-6S) from chondroitin 6-sulfate per minute at 37° C.

Western blot analysis IgG specific to chondroitinase ABC was isolated from antisera raised in guinea pig using the technique described previously (Sato, N. et al. (1988) Biotechnol. Appl. Biochem. 10: 385-393). Proteins in crude cell extracts prepared from E. coli transformant were separated by sodium dodecyl sulfate-polyacrylamide gel electrophoresis (SDS-PAGE) as described previously (Sato, N. et al. (1986) Agric. Biol. Chem. 50: 1057-1059). Western blotting procedures were described previously (Sato, N. et al. (1989) Appl. Microbiol. Biotechnol. 30: 153-159).

### Example 1

Isolation and sequence determination of the chondroitinase ABC gene According to the amino acid sequence of the N-terminal region of purified chondroitinase ABC (Ala-Thr-Ser-Asn-Pro-Ala-Phe-Asp-Pro-Lys-Asn-Leu-Met-Gln-Ser-Glu-Ile-Tyr (FIG. 1-A) (SEQ ID NO:3)), a set of degenerate oligo mixed primers (5′-GCNACNUCNAAYCCNGC-3′ (P-1, sense) (SEQ ID NO:5); 5′-GCNACNAGYAAYCCNGC-3′ (P-2, sense) (SEQ ID NO:6); 5′-UACGUYAGNCUYUADAU-3′ (P-3, antisense) (SEQ ID NO:7); 5′-UACGUYUCRCUYUADAU-3′ (P-4 antisense) (SEQ ID NO:8))(FIG. 1-A) were synthesized as follows. To determine the appropriate primers for sequencing, PCR amplification of a combination of primers P-1 (SEQ ID NO:5), P-2 (SEQ ID NO:6) (sense) and P-3 (SEQ ID NO:7), P-4 (SEQ ID NO:8) (antisense) was performed. After agarose gel electrophoresis of these PCR products, a 54 bp fragment was extracted and directly inserted into pT7 Blue PCR vector, and the inserted fragment was sequenced. The nucleotide sequence of this fragment was found to be identical to the N-terminal amino acid sequence (FIG. 1-B) (SEQ ID NO:3). Then, using primer A (5′-GCAACCAGCAATCCTGCA-3′) (SEQ ID NO:10), primer B (5′-GACTACGTCAGGCTTTTAAT-3′) (SEQ ID NO:11) (FIG. 1-B) and 1 μg of P. vulgaris genomic DNA as a template, PCR analysis was performed and PCR products were analyzed by agarose gel electrophoresis. No non-specific PCR products were observed.

We then diluted λEMBL3 recombinant phage stock library. The diluted library was used for PCR screening. An unique 54 bp fragment was clearly detected until the dilution of 1/10³ (2×10⁵ pfu) phage stock solution as a template. The diluted phage solution was divided by 1/10 (2×10⁴ pfu) and was infected into E. coli P2392. They were then subjected to plaque hybridization using ³²P-labeled probe (5′-CATTTGATCCTAAAAATCTGATGCA-3′) (SEQ ID NO:9)(FIG. 1-B). The recombinant phages were chosen at random and analyzed by restriction mapping and Southern blotting. All phages contained common 4.2 kb EcoRV-EcoRI, 1.1 kb ClaI, and 2.0 kb EcoRV-HindIII fragments which hybridized strongly with the probe (SEQ ID NO:9). The restriction maps of three types of SalI fragments are shown in FIG. 1-C. Southern hybridization patterns of restricted genomic DNA from P. vulgaris matched the restriction map of these fragments. This result suggests that the 4.2 kb EcoRV-EcoRI fragment originated in the P. vulgaris genome, and therefore, the chondroitinase ABC gene exists as a single copy. When purified chondroitinase ABC from P. vulgaris was analyzed by SDS-PAGE, two types of chondroitinase ABC protein, one 100 kd protein and one subunit-like protein at 80 kd and 20 kd, were observed. The amino acid composition of the 100 kd protein and the subunit-like protein (80 kd and 20 kd) were quite similar, and the N-terminal amino acid sequences of the 100 kd and 20 kd proteins were identical. The results indicate that the two forms of chondroitinase ABC were not derived from two separate chondroitinase ABC genes.

The 5.2 kb SalI-EcoRI fragment in the recombinant λEMBL3 (No. 11-5) (FIG. 1-C) was subcloned into pSTV29 for sequencing and the resulting hybrid plasmid was designated pCHS6. The entire 3,063 bp nucleotide sequence of the coding region for the chondroitinase ABC gene as well as 224 and 200 nucleotides of the upstream and downstream regions, respectively, and the deduced amino acid sequence of chondroitinase ABC are shown in FIG. 7 (SEQ ID NO:1). The 25-mer oligonucleotide probe (SEQ ID NO:9) hybridized to nucleotide 314-337. The 16/18 nucleotide of primer A and the 17/18 nucleotide of primer B were the same in nucleotides 297-313 and 333-349. The G+C content of the chondroitinase ABC gene was 38.6%. The open reading frame encoded a polypeptide with a molecular weight of 115,218, which represents a precursor polypeptide containing a signal peptide sequence that is subsequently cleaved off at Ala²⁴-Ala²⁵ during secretion of the mature chondroitinase ABC protein having a molecular weight of 112,365.

### Example 2

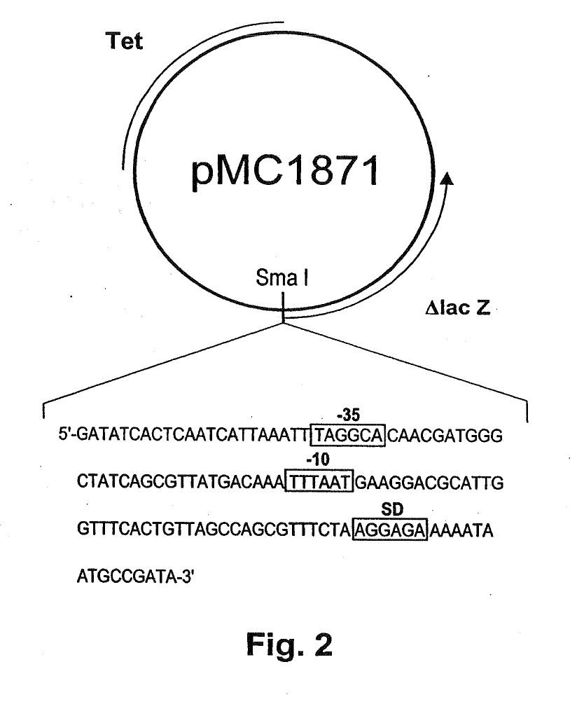

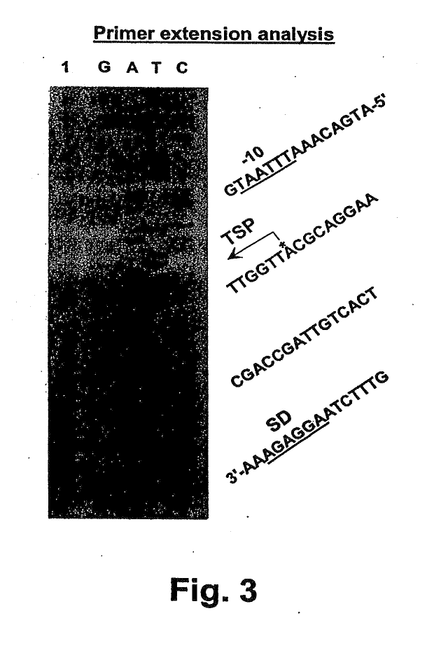

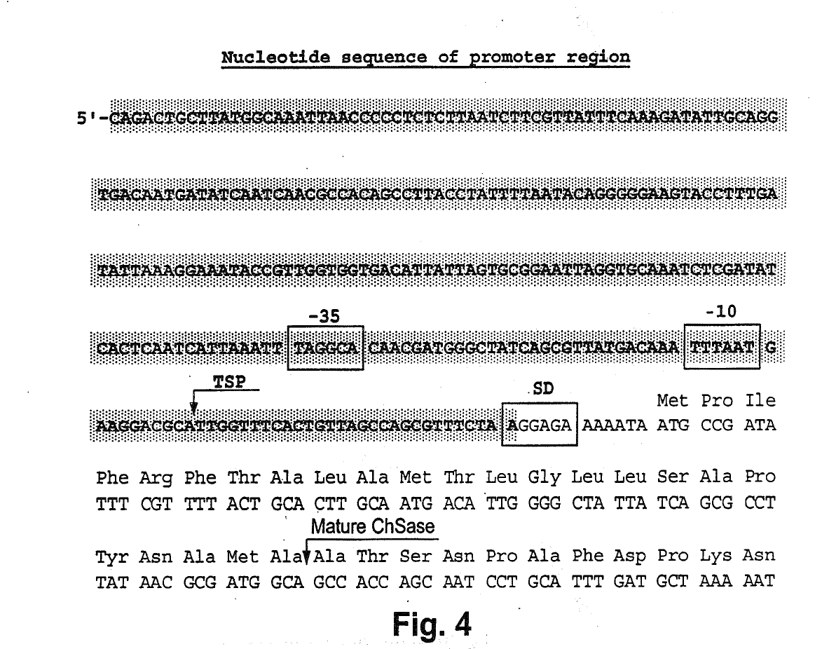

Analysis of the transcription region of the chondroitinase ABC gene In order to confirm the potential promoter region of the chondroitinase ABC gene, we amplified the region of nucleotide 112-283 using PCR. The PCR product was blunt-ended with T4 DNA polymerase and inserted into the SmaI site of the promoter selection vector, pMC1871, and the hybrid plasmid, designated pCHSP, was introduced into E. coli JM109 (FIG. 2) (SEQ ID NO:14). The transformant was then cultured in an LB medium containing 25 μg/ml tetracycline at 37° C. for 14 hr, and β-galactosidase activity was assayed (Table I). Although the β-galactosidase activity of the E. coli transformant carrying pMC1871 was not detectable, the E. coli transformant carrying pCHSP produced β-galactosidase. This result indicates that the chondroitinase ABC gene can function as a promoter in E. coli cells. However, there is a possibility that the promoter recognized in E. coli cells may not be the promoter in P. vulgaris. To confirm that the promoter is recognized in P. vulgaris, primer extension analysis was carried out (FIG. 3) (SEQ ID NO:15). The transcription start point was localized to an adenosine 41 bp upstream from the start codon, ATG (FIG. 4) (SEQ ID NO:16). The potential pribnow box (TTTAAT) (nucleotides 169-174) was located 12 bp upstream from the transcription start point (FIG. 4) (SEQ ID NO:16). However, the −35 consensus sequence was not found near 35 bp upstream of the start point except for 47 bp upstream of the start point (TAGGCA) (FIG. 4) (SEQ ID NO:16). The Shine-Dalgarno ribosomal binding site (AGGAGA) (nucleotides 213-218) was found 9 bp upstream from the initiation codon, ATG (FIG. 4) (SEQ ID NO:16). A terminator-like palindrome sequence consisting of an 11 nucleotide stem with a 4 nucleotide loop structure (stacking energy 24 kcal/mol) was located 9 nucleotides downstream from the stop codon, TGA (FIG. 4) (SEQ ID NO:16). Judging from the secondary structure prediction, this stem-loop structure resembles a ρ-dependent transcription terminator.

TABLE I
β-Galactosidase productivity of E-coli transformants

β-Galactosidase activity

| Strain | Activity | Specific activity/ |
|---|---|---|
| E. coli JM109 | (U/mi-culture) | (U/mg-protein) |
| /pMC1871 | 0 | 0 |
| /pCHSP | 0.2 | 0.4 |

1 U is defined as the amount that produced 1 μmol of α-nitrophenol per h.

### Example 3

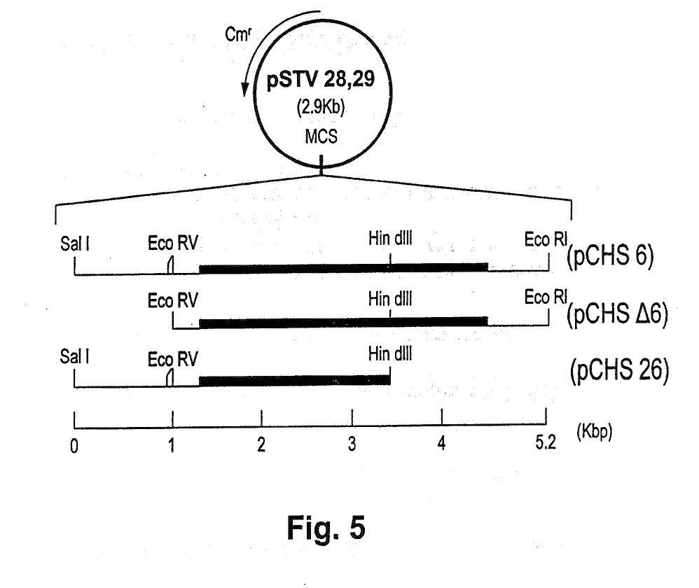

Production of chondroitinase ABC from E. coli transformant To demonstrate that the isolated gene codes for chondroitinase ABC, we constructed pCHSΔ6 and pCHS26 (FIG. 5). pCHSΔ6 was constructed by removing the SalI-EcoRV region (about 1 kb) upstream from the promoter region from the chondroitinase ABC gene. While pCHS26 was constructed by removing the HindIII-EcoRI region which corresponded to about one third of the 3′-terminal region of the chondroitinase ABC structural gene. These plasmids (pCHS6, pCHSΔ6 and pCHS26) were introduced into E. coli XL1-Blue, and E. coli transformants were cultured in chondroitin or glucose medium, and chondroitinase ABC activities were assayed using the crude extract. The culture fluids of the chondroitin medium were also analyzed to determine degradation products of chondroitin 6-sulfate (Table II). The E. coli transformant carrying pCHS6 (containing a 1.0 kb fragment upstream from the promoter) produced the chondroitinase ABC when cultured in chondroitin medium, however, no chondroitinase ABC activity was observed when the transformant was cultured in glucose medium. In contrast, the E. coli transformant carrying pCHSΔ6 produced chondroitinase ABC when cultured in either chondroitin or glucose media. The production levels of chondroitinase ABC, cultured in chondroitin media, were 2.6 fold (/pCHS6) and 187 fold (/pCHSA6) higher than that of P. vulgaris. Even cultured in glucose medium, the production level of chondroitinase ABC in the E. coli transformant carrying pCHSΔ6 was 187 fold higher than that of P. vulgaris cultured in chondroitin medium. This result suggests that the regulatory sequence might be in the SalI-EcoRV region. Although chondroitin 6-sulfate added to the medium was degraded (p/CHS6 and /pCHSA6), E. coli transformants were not able to utilize chondroitin sulfate as a carbon source.

TABLE II
Chondroitinase ABC Activity of E. coli Transformants

| | Intracellular chondroitinase ABC activity | | | | Cultured |
|---|---|---|---|---|---|
| | Chondroitin | | Glucose | | medium |
| | medium (0.3%) | | medium (0.3%) | | Amount of 4,5 |
| | | Specific^{b} | | Specific | Δ chondroitin-6 |
| Strain | Activity^{a} | activity | Activity | activity | (μg/ml-culture) |
| E. coli | 0 | 0 | 0 | 0 | 0 |
| XLI-Blue | | | | | |
| /pSTV29 | 0 | 0 | 0 | 0 | 0 |
| /pCHS6 | 4.1 × 10⁻³ | 1.6 × 10⁻² | 0 | 0 | 192.7 |
| /pCHS26 | 0 | 0 | 0 | 0 | 0 |
| /pCHSΔ6 | 0.3 | 1.2 | 0.3 | 0.5 | 1542.4 |
| P. vulgaris | 1.6 × 10⁻³ | 1.2 × 10⁻² | 0 | 0 | 1738.4 |

^{a}1 U: enzyme activity producing 1 μmol, 4,5Δ chondroitin-6 per min
^{b}U/mg-protein

It has been reported that the Bacteriodes thetaiotaomicron chondroitin lyase II gene is adjacent to the chondrosulfatase gene which may be a part of an operon (Guthrie, E. P. et al. (1987) J. Bacteriol. 169: 1192-1199). These same investigators reported that the promoter for this gene recognized in E. coli may not be the promoter from which the chondroitin lyase II gene is transcribed from in B. thetaiotaomicron (Id.) In fact, a putative open reading frame 12 bp upstream from the initiation codon, ATG, was found in the chondroitinase ABC gene (FIG. 4) (SEQ ID NO:16). However, primer extension analysis revealed that the transcription start point is located 41 bp upstream from the initiation codon in P. vulgaris (FIG. 3) (SEQ ID NO:15). Even though the chondroitinase ABC gene from P. vulgaris cells was also part of an operon, chondroitinase ABC gene was transcribed 41 bp upstream from the initiation codon in P. vulgaris cells.

The secondary structure of chondroitinase ABC was estimated by the method of Chou and Fasman (Annu. Rev. Biochem. 47: 251-276 (1978)). A highly complex region was found between amino acid residues 450 and 850. The pCHS26 lacks one-third of the chondroitinase ABC gene encoding the C-terminal region (amino acid residues 646-1021). Removing this region of the enzyme caused the disappearance of chondroitinase ABC activity (Table II). This result suggests that there might be an active site in this region.

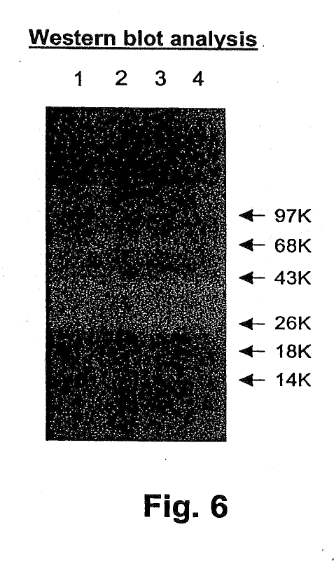

Recombinant chondroitinase ABC produced by E. coli carrying pCHSA6 was analyzed by SDS-PAGE followed by immunoblotting (FIG. 6). The immunoblotting patterns of recombinant and native chondroitinase ABC (100 kd) were quite similar. Our previous report showed chondroitinase ABC purified from P. vulgaris was a subunit structure consisting of a 90 kd and a 20 kd protein by SDS-PAGE (Sato, N. et al. (1986) Agric. Biol. Chem. 50: 1057-1059), because this subunit protein would not be separated even using gel filtration and other chromatographic techniques. However, by analysis of the N-terminal sequence, we found that the 100 kd protein and the 20 kd protein had the same N-terminal amino acid sequence. By immunoblot analysis, the 80 kd protein also reacts with IgG specific to the 100 kd protein. Furthermore, genomic restriction analysis suggested that chondroitinase ABC gene was a single gene. When we extracted the 100 kd band of chondroitinase ABC from the acrylamide gel and electrophoresed it again in SDS-PAGE, 80 kd and 20 kd bands appeared. The purified chondroitinase ABC contained no protease activity. These results suggest that chondroitinase ABC was partially digested not enzymatically, but physically in the course of sample preparation for SDS-PAGE

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims.

1. An isolated nucleic acid encoding chondroitinase ABC, the nucleic acid comprising the nucleotide sequence of SEQ ID NO:1.

2. An isolated nucleic acid encoding chondroitinase ABC, wherein the nucleic acid is obtained from E. coli. XL1-Blue/pCHS5 deposited at Accession NO. FERM BP-4170.

3. An expression vector comprising the nucleic acid of claim 1 or 2 operably linked to a regulatory sequence.

4. A host cell transformed with the expression vector of claim 3.

5. The host cell of claim 4 wherein the cell is eukaryotic.

6. A method of producing chondroitinase ABC protein comprising: culturing the host cell of claim 5 under conditions appropriate for expression; and isolating chondroitinase ABC protein from the culture.

7. An isolated nucleic acid encoding chondroitinase ABC, the nucleic acid comprising a nucleotide sequence which differs from the nucleotide sequence of SEQ ID NO:1, due to degeneracy in the genetic code.

8. An expression vector comprising the nucleic acid of claim 7 operably linked to a regulatory sequence.

9. A host cell transformed with the expression vector of claim 8.

10. A method of producing chondroitinase ABC protein comprising:
culturing the host cell of claim 9 under conditions appropriate for expression; and
isolating chondroitinase ABC protein from the culture.
